(19) United States
(12) Patent Application Publication
(10) Pub. No.: US 2025/0249213 A1
(43) Pub. Date: Aug. 7, 2025

(54) IMPROVEMENTS TO INTRODUCER SHEATHS

(71) Applicant: THREE PEAKS MEDICAL PTY LTD, Norwood (AU)

(72) Inventors: Simon BELCHER, Panorama (AU); Adam DI FIORE, West Hindmarsh (AU); Ronnie EARLES, Bayou Vista, TX (US)

(73) Assignee: THREE PEAKS MEDICAL PTY LTD, Norwood (AU)

(21) Appl. No.: 18/857,466

(22) PCT Filed: May 2, 2023
(86) PCT No.: PCT/IB2023/054537

§ 371 Date: Oct. 17, 2024

(30) Foreign Application Priority Data
May 4, 2022 (WO) PCT/AU2022/050416

(51) Int. Cl. A61M 25/06; A61M 29/02

(52) U.S. Cl. CPC A61M 25/0662; A61M 29/02; A61M 2025/0687; A61M 2205/0216

(57) ABSTRACT

The disclosure generally related to improved introducer sheaths for insertion into the body to provide intravascular access to various medical devices, involving an introducer sheath capable of insertion while providing resistance to frictional forces which may otherwise cause the unintentional compression of the outermost layer of the sheath. Embodiments generally comprise an introducer sheath comprising a circumferentially continuous elastomeric outer layer and a support element. Systems for protecting a luminal surface of a blood vessel are described herein. Also provided herein are methods for the use of these systems. Further, these systems include an elongated sleeve having a proximal opening and a circumferentially continuous elastomeric outer tube, the circumferentially continuous elastomeric outer tube has a smooth inner surface with a substantially round cross-section and includes one or more support elements extending longitudinally from the distal opening towards the proximal opening.

### TECHNICAL FIELD

The technical field generally relates to introducer sheath devices for insertion into the body to provide intravascular access to various medical devices. This includes but is not limited to all arterial and vasculature access, abdominal and thoracic cavities, cerebrospinal, genito-urinary and gynaecological, upper gastrointestinal and colorectal procedures.

Embodiments generally provide improvements to introducer sheaths which resist axial compression of the sheath during insertion while not restricting the radial expansion and/or flexibility of the sheath.

### BACKGROUND

Vascular introducer sheaths are common to intravascular procedures, such as transcatheter aortic valve replacement (TAVR), angioplasty and stenting, to facilitate access to the vascular system for the introduction of removable devices such as wires, balloons, pressure transducers and for the introduction and placement of implantable devices such as mechanical aortic valves and stents.

Vascular introducer sheaths typically consist of a single or dual layer hollow radial cuff or sleeve through which a device may be passed once the introducer sheath is inserted, manoeuvred and placed within the patient's vasculature. The sleeve terminates at one end with a radial collar which is positioned on the patient's skin and within an opening to a blood vessel. The collar typically contains a permeable seal to allow other medical devices to access and withdraw from the vessel while avoiding significant patient blood loss, and may include one or more inlets to allow fluids to be passed into the patient's vascular system through the sleeve cavity.

Where an introducer sheath, or the outermost layer of an introducer sheath, is constructed from elastomeric or other non-rigid materials there is a risk of unintended compression, folding, crumpling, or collapsing of the sleeve during insertion resulting from the shear forces between the insertion point and the outermost layer or between the artery wall and the outermost layer.

For single layer introducer sheaths this can prevent insertion or create a blockage within the artery to prevent the passage of medical devices. For dual layer introducer sheaths this can expose the inner layer on insertion creating additional hazards for the patient such as exposure to sharp edges or damage of the artery from the shear forces between the artery wall and the inner layer.

As a consequence, in such scenarios the patient may experience excessive blood loss during or immediately after surgery, an increase in recovery time, excessive scarring and scar tissue formation at the site of insertion, the need for greater or more prolonged pain management following surgery, or in extreme cases death.

Therefore, there is need for an improvement to such introducer sheaths which prevents axial compression or folding during insertion while not unduly restricting the radial expansion and/or flexibility of the introducer sheath.

It is anticipated that improving introducer sheaths in this way will improve the safety of medical procedures and reduce incidents of failure during insertion of introducer sheaths.

### SUMMARY

In various aspects, embodiments of the present invention relate to an introducer sheath for protecting a luminal surface of a blood vessel, the introducer sheath comprising; an annular collar defining an opening extending through the collar, the annular collar having attached thereto a circumferentially retractable elongated sleeve attached at a proximal opening of the elongated sleeve and extending longitudinally to define a luminal channel terminating at a distal opening of the elongated sleeve, the elongated sleeve comprising a circumferentially continuous elastomeric outer tube having two or more layers comprising an elastomeric layer extending longitudinally and spanning the entire length of the sleeve and a circumferentially discontinuous support layer, wherein the circumferentially discontinuous support layer is formed from one or more support elements extending longitudinally from the distal opening towards the proximal opening, and wherein the elastomeric outer tube has a smooth inner surface with a substantially round or circular cross-section.

This design ensures that inner layers provided in the luminal channel can move smoothly and easily within the outer tube, without being impeded by friction or other forces, such that the inner layer can slide against the inner surface to provide for ease of expanding and retracting. In particular, the smooth and circular nature of the inner surface provides a number of benefits. First, it helps to distribute any forces acting on the element more evenly, reducing the risk of localized wear or damage. Second, it maximises the amount of surface area in contact between the inner layer and the tube, which in turn reduces friction and wear. Finally, it allows for a more consistent and predictable movement of the inner layer within the tube.

The term ‘round’ is to be understood herein as referring to a shape consisting of a curved line that completely encloses a space. The term ‘substantially circular’ is to be understood herein as referring to a shape that closely approximates a perfect circle. In general, a shape that is substantially circular will have a diameter that is uniform throughout and will exhibit no significant bulges, indentations, or irregularities. The term may also encompass shapes that are slightly elliptical or oval, provided that they maintain a high degree of symmetry and overall circularity. For example, a shape is considered to be substantially circular if its deviation from a perfect circle does not exceed a certain predetermined tolerance level. For example, deviation from the average diameter may be always less than 10%, 5%, 2% or 1% of the average thickness of the elastomeric outer tube or less than 0.05, 0.02 or 0.01 mm.

In preferred embodiments, the one or more support elements comprise a substantially crescent shaped profile to provide increased section modulus without increasing thickness, and manufactured from a polymeric material known in the art.

In alternative embodiments, the one or more support elements comprise a wire or strip consisting of any one of stainless steel, nitinol, or another material known in the art to provide tension stiffness. A preferred circumferentially discontinuous support layer may be formed from a combination of support elements, for example a wire or strip selected from those providing tension stiffness in combination with a crescent shaped polymeric material.

In alternative embodiments, the one or more support elements is corrugated at its outer surface to produce substantially stiff ridge portions and elastic groove portions in-between. This configuration allows each support member to flex such that the radius of the one or more support elements can dynamically change with the radius of the elastomeric layer.

The term ‘layer’ is to be understood herein as a sheet or similar form of construction that may partially or entirely cover another article or object. The term ‘layer’ is not to be unduly limited in its construction so as to imply the presence of one or more other layers, articles or objects. For example, an article may comprise two or more layers that may be bonded at their edges rather than in a laminated or layered fashion and may still be regarded as two individual layers in their own right.

The term ‘proximal’ is to be understood herein as defining a location situated close to or at a point of attachment, while the term ‘distal’ is to be understood to be situated far from or at an opposite end to a point of attachment. In the context of the elongated sleeve of an introducer sheath, the point of attachment is the annular collar.

In embodiments, the circumferentially discontinuous support layer may be bonded to the elastomeric layer and/or bonded about the distal opening. The term ‘bonded’ is to be understood herein as defining an arrangement in which two or more discrete objects are joined, connected, or linked such that they share a common operation, act as a single object, or take on common properties. This may be through embedding, adhesion, welding, stitching, compression, friction, electromagnetism, or some other mechanism known to those in the art.

For the purposes of illustration, the breadth of the term ‘bonding’ as used herein includes, but is not limited to, bonding by interference fit, friction fit or that achieved by other mechanical locking features (e.g. utilising a process which temporarily increases the elastomers size). The term ‘circumferential retraction’ is to be understood herein as defining the ability of an object to retract its circumference from an expanded size to either its initial circumference or a circumference therebetween.

In preferred embodiments, the inner surface of the elastomeric outer tube is partially formed by the elastomeric layer and partially formed by the support layer.

The elongated sleeve preferably comprises a coiled expandable inner sheet positioned longitudinally within a lumen formed by the circumferentially continuous elastomeric outer tube, positioned substantially in contact with a luminal surface of the circumferentially continuous elastomeric outer tube.

The one or more support elements preferably extend longitudinally, span the entire length of the sleeve and are bonded to an inner luminal surface of the continuous elastomeric layer, whereby the circumferentially discontinuous support layer is configured to resist longitudinal compression of the circumferentially continuous elastomeric outer tube along the entire length of the sleeve.

Preferably, the one or more support elements is formed of a semi-rigid material. The term ‘semi-rigid’ is to be understood herein as defining a material or structure that is somewhat flexible but has a certain degree of stiffness or rigidity, as opposed to being completely flexible or completely rigid. Preferably, the one or more support elements is more rigid than the elastomeric element. More preferably, the one or more support elements is formed of polyurethane.

Preferably, the one or more support elements is formed of a biocompatible material and has a k approximately equal to or greater than 0.1, wherein k is expressed by the formula:
k=F/δ/l, wherein;
k=longitudinal stiffness in Newtons per millimetre per metre (N/mm/m)
F=applied longitudinal force in Newtons (N)
δ=extension, deflection in millimetres (mm)
l=length of support element in metres (m).

Preferably, the one or more support elements has a k approximately equal to or greater than 2N/mm/m. More preferably, the one or more support elements has a k approximately equal to or greater than 4N/mm/m.

The term stiffness according to the formula above, describes a ratio between a force applied to a body, to the resultant deflection through that direction of force. This deflection may be due to elastic or inelastic extension or compression of the material forming the body or crumpling or distortion of the geometry of the body.

Preferably, the one or more support elements comprises a biocompatible material selected from the group consisting of: polyurethane, polyolefin, polypropylene, polyethylene, styrenic polymer, polyester, polycarbonate, fluorinated polymer, polyamide, polyether block amide, polyoxymethylene, polyimide, polyetherimide, polyether ether ketone, titanium and titanium alloy, stainless steel, including alloys and blends. It could also be made from fibrous or woven materials including cotton, carbon, aramid, polyamide and polyester.

Preferably, the circumferentially continuous elastomeric outer tube is formed of biocompatible materials and has a kr approximately equal to or greater than 1.5, wherein kr is expressed by the formula:
kr=ki/k0=(F*δ0)/(F*δi), wherein;
kr=stiffness ratio of improved elastomeric layer to original
k0=original elastomeric layer longitudinal stiffness (N/mm/m)
ki=improved elastomeric layer longitudinal stiffness (N/mm/m)
F=applied longitudinal force (N)
δ0=original elastomeric layer extension, deflection (mm)
δi=improved elastomeric layer extension, deflection (mm, in).

Preferably, the circumferentially continuous elastomeric outer tube has a kr approximately equal to or greater than 2.5. More preferably, the circumferentially continuous elastomeric outer tube has a kr approximately equal to or greater than 3.5.

Longitudinal stiffness is to be understood herein to define a ratio between a force applied to the length of a body, to the resultant deflection along that length.

Practical comparison of the shear stiffness of a support element to that of an elastomeric layer entails the application of an equal shear force to each, where the shear force is comparable to that which is encountered during insertion of an introducer sheath into an incision and measuring the deflection of each to produce a ratio, as described in the formula above.

Preferably, the circumferentially continuous elastomeric outer tube comprises a biocompatible material selected from the group consisting of: silicone, isoprene, latex and latex substitute rubber, and other thermoplastic elastomers.

Preferably, the one or more support elements comprises a material selected from the group consisting of fibrous materials, textiles, metals, metal alloys, polymers, mixtures or composites thereof, the selected material having at least 50 times the modulus of the continuous elastomeric layer. For example, measured according to ASTM D638 or ASTM D412 or ASTM D792.

More preferably, the one or more support elements comprises a material having at least 200 times the modulus of the continuous elastomeric layer. More preferably still, the one or more support elements comprises a material having approximately 300 times the modulus of the continuous elastomeric layer.

In further aspects, embodiments of the present invention relate to a system for protecting a luminal surface of a blood vessel, the system comprising an introducer sheath being formed from; an annular collar defining an opening extending through the collar, the annular collar having attached thereto a circumferentially retractable elongated sleeve attached at a proximal opening of the elongated sleeve and extending longitudinally to define a luminal channel terminating at a distal opening of the elongated sleeve, the elongated sleeve comprising a circumferentially continuous elastomeric outer tube having two or more layers comprising a continuous elastomeric layer extending longitudinally and spanning the entire length of the sleeve and a circumferentially discontinuous support layer, the elongated sleeve comprising a coiled expandable inner sheet positioned longitudinally within a lumen formed by the circumferentially continuous elastomeric outer tube, positioned substantially in contact with a luminal surface of the circumferentially continuous elastomeric outer tube, wherein the circumferentially discontinuous support layer is formed from one or more support elements extending longitudinally from the distal opening towards the proximal opening, and wherein the elastomeric outer tube has a smooth inner surface with a substantially circular cross-section.

Preferred systems further comprise a dilator having a dilator tip of greater diameter than the distal opening of the elongated sleeve, wherein the dilator tip is configured to pass through a lumen formed by the expandable inner sheet along the length of the circumferentially retractable elongated sleeve.

The one or more support elements preferably extend longitudinally, span the entire length of the sleeve from the distal opening, or near the distal opening, to the proximal opening, or as close to the proximal opening as practicable and are bonded to an inner luminal surface of the continuous elastomeric layer, whereby the circumferentially discontinuous support layer is configured to resist longitudinal compression of the circumferentially continuous elastomeric outer tube along the entire length of the sleeve.

In alternative embodiments the one or more support elements may extend partially along a length of the sleeve and may be circumferentially discontinuous along its length. Alternatives to bonding may include adhesives, stitching, material reflow, co-extrusion, encapsulation (or lamination), or other such methods known in the art to increase friction therebetween.

The one or more support elements is preferably formed of a biocompatible material and has a k approximately equal to or greater than 1, wherein k is expressed by the formula:
k=F/δ/l, wherein;
k=longitudinal stiffness in Newtons per millimetre per metre (N/mm/m)
F=applied longitudinal force in Newtons (N)
δ=extension, deflection in millimetres (mm)
l=length of support element in metres (m).

The circumferentially continuous elastomeric outer tube is alternatively formed of a biocompatible material and has a kr approximately equal to or greater than 1.5, wherein kr is expressed by the formula:
kr=ki/k0=(F*δ0)/(F*δi), wherein;
kr=stiffness ratio of improved elastomeric layer to original
k0=original elastomeric layer longitudinal stiffness (N/mm/m)
ki=improved elastomeric layer longitudinal stiffness (N/mm/m)
F=applied longitudinal force (N)
δ0=original elastomeric layer extension, deflection (mm)
δi=improved elastomeric layer extension, deflection (mm, in).

The one or more support elements preferably comprises a material selected from the group consisting of fibrous materials, textiles, metals, metal alloys, polymers, mixtures or composites thereof, the selected material having at least 50 times the modulus of the continuous elastomeric layer.

The support element should have a tensile and flexural modulus that is higher than the elastomeric material to provide the required structural improvement. As the modulus difference increases, the required size of the support element decreases, meaning that the impact of the support on the radial expansion stiffness is reduced. For example, a support element made from titanium can be much smaller in cross section than a polyurethane support element for the same longitudinal stiffness effect. The support element also needs to have some flexibility to allow the device to curve through the patient anatomy. There are also considerations on buckling resistance-a small stainless steel wire would have very good tensile extension resistance, but would need to be sized to maintain adequate compression resistance. From that perspective the support element material could have a modulus that is 10.000× the elastomer modulus (e.g. 40 A TPE at 0.2 MPa to Stainless Steel at 210 GPa).

In further aspects, embodiments of the present invention relate to a method comprising the steps of; obtaining an introducer sheath being formed from an annular collar defining an opening extending through the collar, the annular collar having attached thereto a circumferentially retractable elongated sleeve attached at a proximal opening of the elongated sleeve and extending longitudinally to define a luminal channel terminating at a distal opening of the elongated sleeve, the elongated sleeve comprising a circumferentially continuous elastomeric outer tube having two or more layers comprising a continuous elastomeric layer extending longitudinally and spanning the entire length of the sleeve and a circumferentially discontinuous support layer bonded to the continuous elastomeric layer and being formed from one or more support elements, the support layer bonded about the distal opening and extending longitudinally therefrom towards the proximal opening, obtaining an expandable inner sheet, positioning the expandable inner sheet longitudinally within a lumen formed by the circumferentially continuous elastomeric outer tube and positioned substantially in contact with a luminal surface of the circumferentially continuous elastomeric outer tube, whereby the circumferentially discontinuous support layer is configured to resist longitudinal compression of the circumferentially continuous elastomeric outer tube.

Preferred methods may further comprise the step of; obtaining a dilator having a dilator tip of greater diameter than the distal opening of the elongated sleeve, passing the dilator tip through a lumen formed by the expandable inner sheet and along the length of the circumferentially retractable elongated sleeve.

Preferred methods may further comprise the step of; passing the dilator tip and introducer sheath through a blood vessel wall.

Embodiments are designed as such to provide an introducer sheath capable of insertion into the blood vessel without crumpling, folding, or compressing in opposition to the direction of insertion, while still allowing for radial expansion and/or tangential flexing.

The functionality of embodiments is dependent on a novel design, whereby a support element, or spine, is incorporated into the circumferentially continuous elastomeric outer tube of the retractable elongated sleeve and extends longitudinally. The continuous elastomeric layer is the outermost layer of the elongated sleeve, through which an introducer or dilator is passed, and preferably maintains the ability to bend during insertion.

Crumpling, folding, or compression of the elongated sleeve may result in a failed insertion and therefore the incorporation of an axially resistive support element into the circumferentially discontinuous support layer results in improved functionality of improved introducer sheaths.

Introducer sheaths preferably offer a balance of characteristics including sufficient bendability to navigate a network of vascular channels, sufficient rigidity to prevent kinking and buckling, and sufficient resilience to retract when circumferentially expanded, or when inadvertently bent or kinked.

The design concept, according to embodiments, is provided by a combination of a continuous elastomeric layer and a circumferentially discontinuous support layer that imparts complementary physical characteristics that cannot be provided by any single component. In particular, the circumferentially discontinuous support layer of embodiments provides rigidity while the circumferentially continuous elastomeric outer layer provides resilience, and each provide a sufficient degree of bendability to navigate a patient's vasculature.

In preferred embodiments of the invention the one or more support elements are substantially linear longitudinally.

Longitudinal is to be understood herein as defining a direction through the luminal channel defined by the proximal and distal opening, from the proximal end opening to the distal end opening or from the distal end opening to the proximal end opening.

The term ‘radial direction’ is to be understood herein as defining a direction which is substantially perpendicular to the axial direction.

The term ‘substantially linear’ is to be understood herein as defining a construction which is largely linear but may contain some non-linear elements which do not affect the overall directionality and shape on a macro scale.

The term ‘substantially in contact’ is to be understood herein as defining an assembly in which a significant portion of a surface is in contact with another surface, however, contact with an entirety of each surface is not required.

In preferred embodiments, the expandable inner sheet is an expandable inner layer formed from the coiling of a single sheet upon itself and characterised as having an outward annular resistance.

The expandable inner sheet may comprise cut outs, slits and other sections to improve or alter the physical properties of the elongated sleeve, in particular, the bendability, resistance to kinking or resilience of the elongated sleeve.

The expandable inner sheet may be integrally formed from a substantially uniform stiff polymeric material comprising a notch or cut-out. The expandable inner sheet may be configured to coil in an overlapping arrangement in its relaxed state and to coil helically about the longitudinal axis of the circumferentially retractable elongated sleeve.

In certain embodiments, the support element may extend partially from a distal end of the of the circumferentially retractable elongated sleeve to a proximal end of the of the circumferentially retractable elongated sleeve. It may extend therebetween intermittently or continuously.

In various embodiments, the profile of the circumferentially continuous elastomeric outer tube along the length of the circumferentially retractable elongated sleeve may be of a constant radius or a dynamic radius, whereby the sleeve is formed as tapering or as a tapering portion, in an axial direction.

In preferred configurations and embodiments, the support element is a substantially straight extrusion running longitudinally from a distal end of the of the circumferentially retractable elongated sleeve to a proximal end of the of the circumferentially retractable elongated sleeve.

The support elements may be manufactured from the same or similar material as the circumferentially continuous elastomeric outer tube, with a varied consistency or density, it may be manufactured from the same material as the expandable inner layer, or it may be manufactured from a metallic substrate, or other material.

In alternative embodiments, the circumferentially continuous elastomeric outer tube may be optimised for both containment of the support element, or the smooth insertion of the introducer sheath into a patient.

In preferred embodiments, the support element may be bonded to the inner surface of the circumferentially continuous elastomeric outer tube and extends longitudinally along the length of the circumferentially retractable elongated sleeve from a distal end of the of the circumferentially retractable elongated sleeve to a proximal end of the of the circumferentially retractable elongated sleeve. It may extend therebetween intermittently or continuously.

In preferred embodiments, the expandable inner sheet is a helically coiled sheet of stiff polymeric material configured to coil in an overlapping arrangement in its relaxed state and to coil helically about the longitudinal axis of the circumferentially retractable elongated sleeve.

In alternative embodiments and configurations, the addition of the expandable inner sheet may be applied to any of the alternative embodiments and configurations of the circumferentially continuous elastomeric outer tube.

In preferred embodiments, the expandable inner sheet expands with the insertion of the dilator or surgical instruments within the lumen of the circumferentially retractable elongated sleeve. The expansion of the expandable inner sheet causes the subsequent expansion of the circumferentially continuous elastomeric outer tube at the portion which is not reinforced by the circumferentially discontinuous support layer.

In further aspects, embodiments relate to a method of use of an introducer sheath comprising the steps of obtaining an introducer sheath according to any one of the embodiments defined herein, passing a rigid introducer or dilator through the luminal channel of the introducer sheath, and introducing the introducer sheath into a blood vessel.

In further aspects, embodiments relate to a method of manufacture of an introducer sheath comprising the steps of obtaining a rigid collar and a continuous elastomeric layer according to preferred embodiments of the invention and attaching the continuous elastomeric layer at the proximal opening with the rigid collar.

In further aspects, embodiments relate to a method of manufacture which may comprise the additional steps of obtaining a second sleeve layer according to preferred embodiments of the invention, attaching the second sleeve layer at the proximal opening with the rigid collar, and placing the second sleeve layer within the continuous elastomeric layer.

### BRIEF DESCRIPTION OF THE FIGURES

FIGS. 1(a) and 1(b) provide front perspective views of prior art introducer sheaths wherein 1(a) provides a front perspective view of a two-layer, composite introducer sheath and 1(b) provides a front perspective view of a single layer sheath.

FIGS. 2(a) to 2(c) provide side section views at three stages of the prior art problem wherein 2(a) shows an introducer sheath known in the art prior to insertion, 2(b) shows the introducer sheath and the skin-body interface during insertion, and 2(c) shows the resulting crumpling which takes place at the skin surface following insertion.

FIG. 3 provides a front perspective view of an introducer sheath according to an embodiment of the invention.

FIGS. 4(a) and 4(b) provide a detailed front perspective view according to an embodiment of the invention wherein 4(a) illustrates improvement to an outer layer of an embodiment showing and 4(b) illustrates the improved outer layer in conjunction with an inner layer.

FIG. 5 provides a detailed section view of the embodiment illustrated at FIG. 4(a) through lines A-A.

FIGS. 6(a) to 6(c) provide a detailed side section view of various embodiments of the invention wherein 6(a) illustrates a support element ‘in-line’ with the outer layer of the sleeve, 6 (b) illustrates a support element ‘external’ to the outer surface of the sleeve, and 6(c) illustrates a support element ‘laminated’ within the outer layer of the sleeve.

FIGS. 7(a) to 7(c) provide side section views at three stages during the use of a sleeve according to an embodiment of the invention wherein 7(a) shows an introducer sheath prior to insertion, 7(b) shows the improved introducer sheath and the skin-body interface during insertion, and 7(c) shows the smooth passage of the outer layer of the improved introducer sheath through the skin following insertion.

FIG. 8 provides a front perspective view of an alternative embodiment of the invention having multiple support elements.

Several embodiments of the invention are described in the following examples.

### DETAILED DESCRIPTION OF EMBODIMENTS

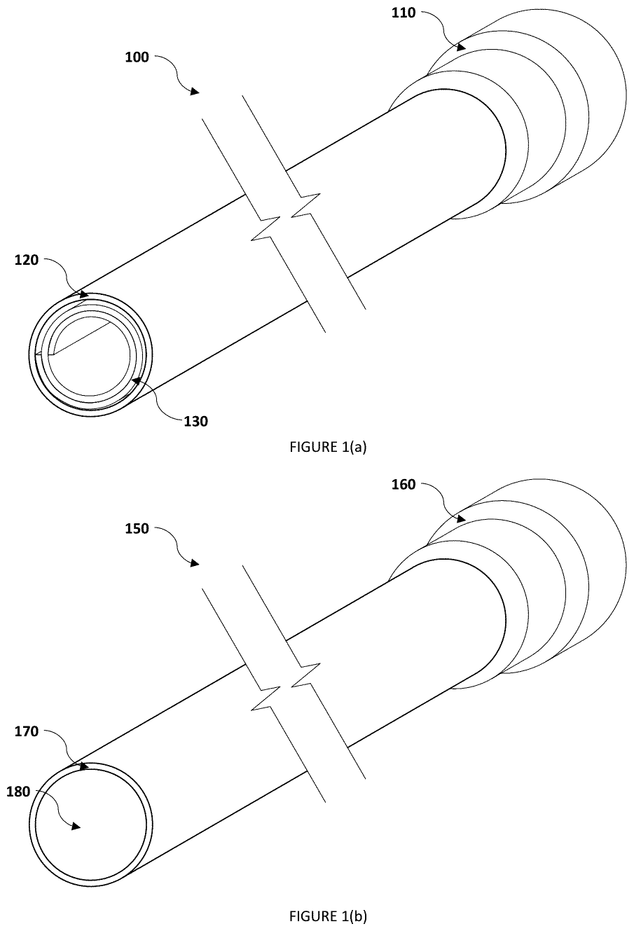

With reference to FIG. 1, introducer sheaths known in the prior art take one of two general forms. FIG. 1 (a) illustrates a two layered introducer sheath 100 known in the art. Such two layered introducer sheaths are composed of three main components, a collar 110, an outer layer 120 and an inner layer 130. The two layered introducer sheath illustrated at FIG. 1(a) is composed of a coiled inner layer 130, which permits the radial expansion and retraction of the introducer sheath 100, loosely coiled within an elastomeric outer layer 120, such that each layer can slide against the other to provide for ease of movement within a vessel. Either or both of outer layer 120 or inner layer 130 may be bonded to collar 110. Collar 110 provides an opening for introducing a dilator within the lumen of the inner layer 130.

FIG. 1(b) illustrates an alternative single layered introducer sheath 150 known in the art broadly constructed of two main components; a rigid annular collar 160 and an elongated sleeve 170. Collar 160 is a hollow structure bonded to the elongated sleeve 170. Collar 160 allows materials to be introduced into the inner lumen 180 of sleeve 170 through an opening of the sleeve. Annular collar 160 is formed of a rigid material to allow a user to handle the collar and pass an object or material therethrough into inner lumen 180. It terminates in an opening of a similar diameter to the opening of sleeve 170 to enable sleeve 170 to be placed and secured therein, thereby joining the two components.

Both forms of prior art introducer sheaths are formed of an elongated flexible tube attached to collar. The form of introducer sheaths 100 and 150 is maintained during insertion of the introducer sheath into a vessel by a dilator which is placed within the introducer sheath prior to use, and the introduction of medical instruments therethrough.

As the outer surface of an introducer sheath must be smooth and flexible to avoid catching on or tearing of fragile blood vessels, in both instances the outer layer 120 and the sleeve 170 may be formed of materials that are subject to folding or crumpling upon insertion; creating the potential for a failed insertion.

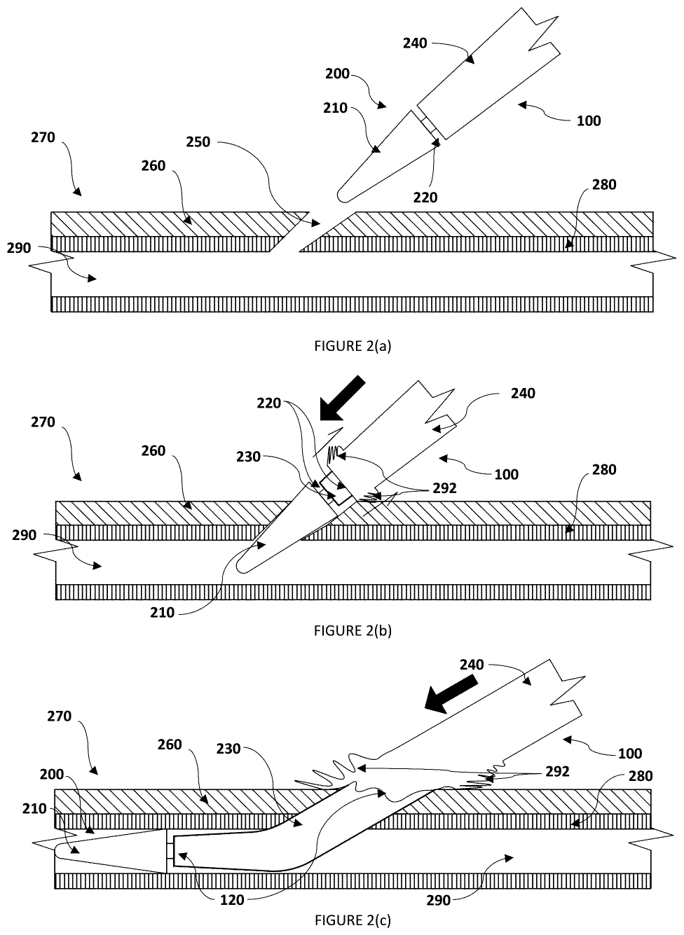

FIGS. 2(a) to 2(c) illustrate the problem caused by prior art devices in greater detail. While FIG. 2 illustrates the problem with respect to the prior art devices of the two layer form, the problem is present for all introducer sheaths being formed of a smooth and flexible outer material. FIG. 2(a) illustrates the prior art introducer sheath of FIG. 1(a) prior to insertion. Prior to its introduction into a vessel, dilator 200 is inserted through collar 110 (not shown) so that dilator tip 210 extends through distal opening 220 and is positioned optimally against inner layer 230 and outer layer 240. An opening or incision 250 is made in the skin 260 of patient 270 to provide access for introducer sheath 100 and dilator 200 through artery wall 280 into artery 290.

FIG. 2(b) illustrates the prior art introducer sheath of FIG. 1(a) during insertion and at the time of engagement with incision 250 and skin 260. Dilator tip 210 is inserted into the incision 250 to thereby expand the opening enough for introducer sheath 100 to access artery interior 290. During insertion, outer layer 240 contacts skin 260 and the internal surfaces of incision 250. Friction against outer layer 240, and the elasticity of the material which forms outer layer 240, results in the crumpling, compressing and folding of the outer layer 240 in the direction from the distal opening 220 towards the proximal end(not shown). This crumpling 292 exposes the inner layer 230. Sharp edges formed at the distal opening typically catch and cause damage to the incision 250 or artery wall 280 during insertion.

FIG. 2(c) illustrates the prior art introducer sheath of FIG. 1(a) during insertion into artery 290. Once crumpling of the outer layer 240 takes place, inner layer 230 is exposed. Dilator tip 210 continues to progress into the artery interior 290 with introducer sheath 100 following. Crumpled outer layer 292, 240 causes damage to the artery wall 280 from the sharp edges of inner layer 230 at distal opening 220, or from catching of the crumpled outer layer 292, 240 at the artery wall 280. Such failed insertions require use of a new introducer sheath for another attempt at introducer sheath insertion, which in turn introduces further risk to the patient.

The problem illustrated in FIGS. 2(a) to 2(c) also applies to single layer introducer sheaths of the kind illustrated in FIG. 1(b). While there is no exposure of a sharp inner layer, any crumpling or folding of the single layer sleeve could lead to a sub-optimal insertion, blockage, and subsequent insertion failure.

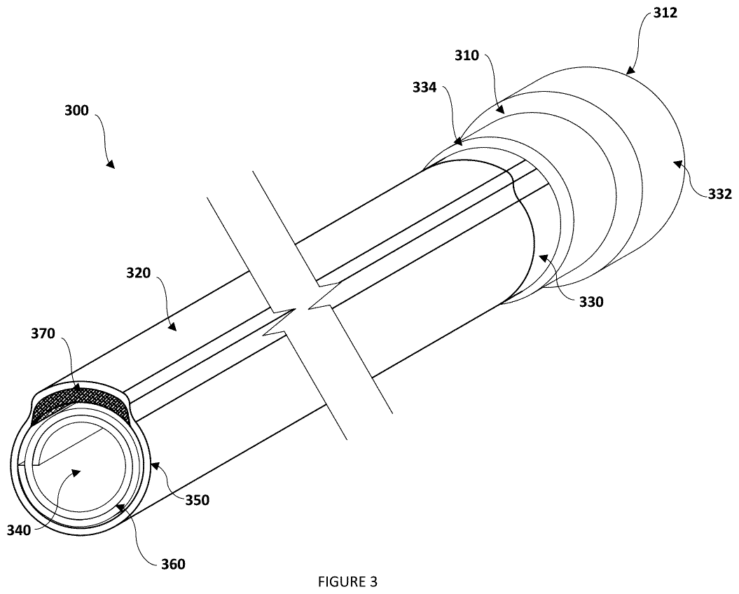

FIG. 3 illustrates an improved introducer sheath according to embodiments of the invention. Introducer sheath 300 is generally constructed of two main components; a rigid annular collar 310 bonded to an elongated, sleeve 320 at the sleeve's proximal end 330. Collar 310 is a hollow, rigid structure and is constructed to allow materials to be fed therethrough by a user, into the inner lumen of sleeve 320 through an opening in the sleeve at its proximal end 330. Collar 310 may comprise inlets (not shown) to allow fluids to be passed into the lumen of the introducer sheath 300, and is formed of a rigid material to allow a user to handle the collar and pass an object or material therethrough via opening 312 formed at the proximal end 332 of collar 310. Collar 310 terminates at a distal end 334 in a narrowed opening of a similar diameter to the opening of sleeve 320 at the sleeve's proximal end 330. Similarity of the diameter of the distal end 334 of collar 310 and sleeve proximal end 330 enables sleeve 320 to be placed and secured within collar 310, thereby joining the two components together.

Sleeve 320 extends away from the proximal end 330 of collar 310 at critical points along the length of sleeve 320 for ease of insertion into an incision made to access the patient's vascular system. Sleeve 320 is a smooth, flexible structure tapering, in part, between proximal end 330 and distal end 340 to ease the insertion and navigation of the sheath within a vessel. Sleeve 320 is expandable and retractable to enable a wider dilator, valve or other medical device to be introduced into the patient through the lumen of the sleeve 320; which is capable of expanding around the device, and capable of gently pressing against the luminal wall of the vessel as it travels through the vessel; and then also to retract to a size similar to its original size once the device is removed, so that it can be gently removed from the patient.

Sleeve 320 is formed of two thin, flexible layers; elastomeric outer tube 350 and expanding inner layer 360. Outer elastomeric tube 350 is formed as an elongated tube maintaining within it a coiled expandable inner sheet forming expanding inner layer 360. Outer elastomeric tube 350 is improved over the prior art by the addition of a semi-rigid support layer taking form as a widened semi-rigid support element 370.

Outer elastomeric tube 350 is formed from an elastomeric material that can be stretched when pressure is exerted from within the lumen of sleeve 320 but also substantially returns sleeve 320 to its original diameter once internal pressure is relaxed. In a relaxed state, outer elastomeric tube 350 has a wall thickness of less than 0.4 mm, and preferably less than 0.2 mm.

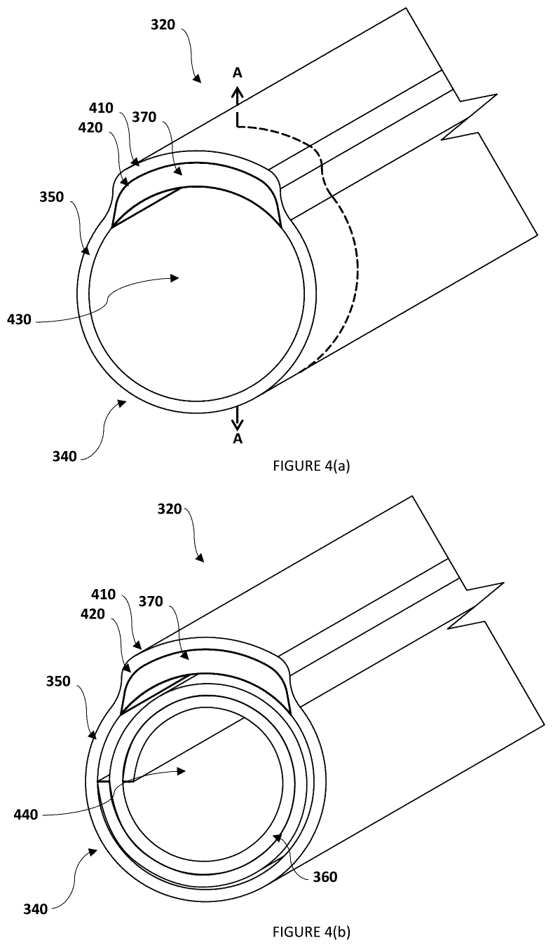

With reference to FIGS. 4(a) and 4(b), outer elastomeric tube 350 is formed from multiple layers including an elastomeric layer 410 and a semi-rigid support layer 420. Elastomeric layer 410 is formed from silicone to maximise the elasticity and minimise wall thickness and thereby minimise the risk of damage to the vascular system. However, other materials may be selected by those skilled in the art for this purpose, by assessing potential materials and selecting those that have suitable properties. Suitable materials may include materials such as latex rubber or non-latex substitutes including nitrile rubber, polyvinylchloride, neoprene, polypropylene and polyisoprene, polyurethane, other thermoplastic elastomers and the like.

Elastomeric layer 410 is formed from an extrusion process and is shaped through this process to receive semi-rigid support element 370 within a groove formed lengthwise within an internal luminal surface 430 of elastomeric layer 410. Elastomeric layer 410 and semi-rigid support member 370 are formed into complementary shapes and bonded together by co-extrusion, such that the resulting outer elastomeric tube 350 is formed from the assembled and bonded elastomeric layer 410 and semi-rigid support element 370.

Semi-rigid support element 370 is constructed from a polymeric material. It is formed in a substantially crescent or D shape and is contained within a complementary shape formed by elastomeric layer 410. The shape and semi-rigid support member extend longitudinally from the distal end 340 towards the collar 310. In preferred embodiments, semi-rigid support element 370 extends wholly from the distal end 340 to collar 310 without interruption.

Outer elastomeric tube 350 maintains the expanding inner layer 360 in a coiled form within the lumen of the outer elastomeric tube 350 in contact with and applying forced outwardly onto the internal luminal surface 430 of elastomeric layer 410. Once coiled and positioned within the internal luminal surface 430 of elastomeric layer 410, the outer elastomeric tube 350 and inner layer 360 form sleeve lumen 440 to produce a composite sheath.

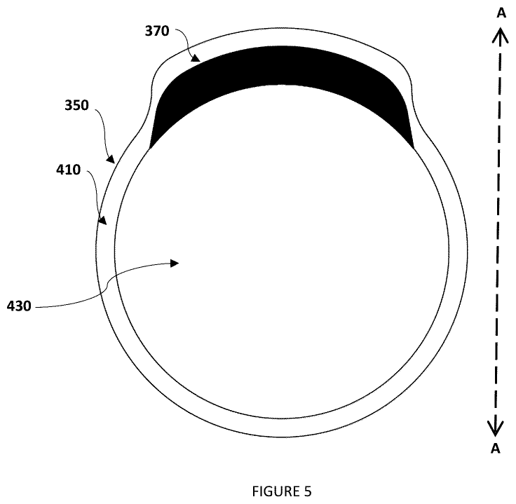

FIG. 5 provides a section of outer elastomeric tube 350 along lines A-A shown in FIG. 4(a). With reference to FIG. 5, semi-rigid support element 370 has a thickness of between 0.1 mm and 0.5 mm and is shaped by coextrusion to fill a position in-line with the generally circular profile of outer elastomeric tube 350; such that internal luminal surface 430 of elastomeric layer 410 is unabridged and complete. In this form the profile of the outer elastomeric tube 350 protrudes outwards with the profile of semi-rigid support element 370 protruding lengthwise. In this form, the outermost surface of semi-rigid support element 370 is encompassed by elastomeric layer 410 of a thinner or similar thickness as the remainder of elastomeric layer 410.

FIG. 4(a) illustrates a preferred configuration and embodiment, further detailed in FIG. 5, in which outer elastomeric tube 350 is substantially circular in profile having a smooth consistent circular internal surface and a protuberance along its length of its external surface corresponding to the shape of the semi-rigid support element 370. As shown in FIGS. 4(a) and 4(b), the outermost surface of outer elastomeric tube 350 transitions from a smooth cylindrical shape to a smooth taper, fillet, or chamfer such that the protuberance in the external surface of the outer elastomeric tube 350 does not interfere with the incision or artery during insertion.

The outermost surface of outer elastomeric tube 350 is of uniform thickness such that it extends over the semi-rigid support element 370 exposing its profile on the outermost surface of outer elastomeric tube 350.

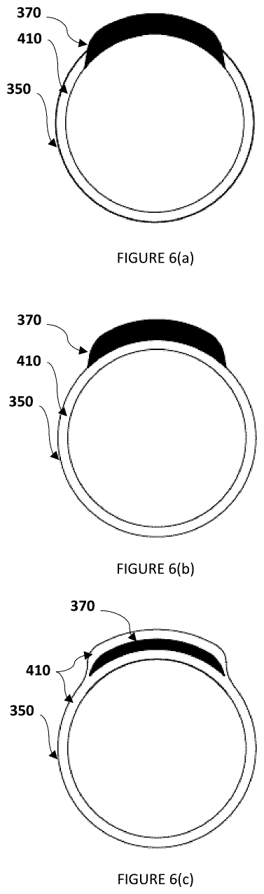

A range of alternative shapes and configurations may be selected by skilled persons depending on the desired characteristics of the elastomeric material or the semi-rigid support element or the ease of manufacture. Particularly useful configurations are illustrated in FIGS. 6(a), 6(b) and 6(c).

FIG. 6(a) illustrates an alternate construction of elastomeric layer 410 and semi-rigid support element 370 wherein semi-rigid support element 370 remains substantially crescent shaped and of an inner radius to correspond with the inner radius of elastomeric layer 410 such that the combination of these two components results in a substantially circular profile, however, the portion of elastomeric layer 410 which protrudes in the preferred embodiment of FIG. 5 is not present. This alternate construction is formed by bonding or affixing each of the side edges of semi-rigid support element 370 which are in contact with elastomeric layer 410 with elastomeric layer 410 to form a circumferentially continuous outer elastomeric tube 350. In this configuration the layers are not laminated or bonder on their upper or lower surfaces.

FIG. 6(b) illustrates an alternative embodiment of elastomeric layer 410 and semi-rigid support element 370 wherein semi-rigid support element 370 is again substantially crescent shaped but of an inner radius to match the outer radius of elastomeric layer 410 and affixed to its outer surface. This embodiment therefore requires the inner surface of semi-rigid support element 370 which is in contact with elastomeric layer 410 to be affixed thereto or formed therewith to produce outer elastomeric tube 350.

FIG. 6(c) illustrates an alternative construction of elastomeric layer 410 and semi-rigid support element 370 wherein semi-rigid support element 370 remains substantially crescent shaped, however, it is laminated between two elastomeric layers 410. Outer elastomeric tube 350 has a substantially circular external profile which is modified such that a portion of the profile has a protuberance at a larger radius than the remaining portion of the profile. Outer elastomeric tube 350 has a smooth, substantially circular, continuous internal profile. Semi-rigid support element 370 is contained between an outermost elastomeric layer 410 and an innermost elastomeric layer 410 and within the protuberance in the external surface of outer elastomeric tube 350.

The encapsulation or lamination of semi-rigid support element 370 within the material of outer elastomeric tube 350 allows for the assembly of the two components without requiring bonding or affixing. This form of construction is especially advantageous in instances wherein the bonding of semi-rigid support elements 370 or elastomeric layer 410 may not be possible; as the semi-rigid support elements may be held in the desired position by lamination or pressure rather than bonding. Such instances may occur where materials are chemically incompatible with bonding agents (e.g. where a metallic semi-rigid support element is used) or wherein bonding increases costs or difficulty of manufacture.

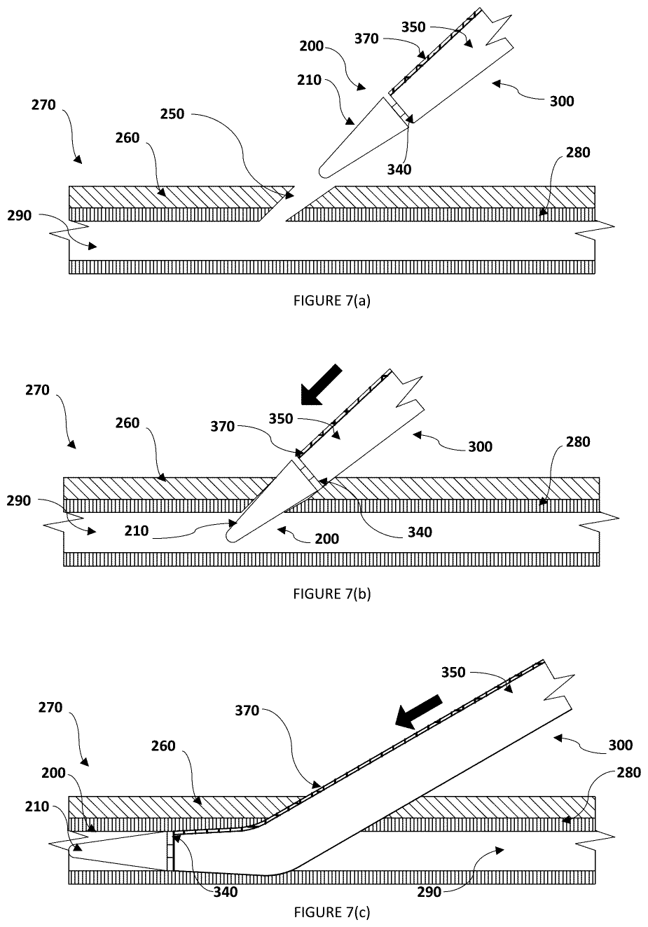

FIGS. 7(a), 7(b), and 7(c) illustrate the resolution of the prior art problem by the embodiment described herein. FIG. 7(a) illustrates the improved introducer sheath described herein prior to insertion. Dilator 200 is inserted through collar 310 and dilator tip 210 extends through distal opening 220 and is positioned to abut against inner layer 230 and outer layer 240, whereby it is supported in position by semi-rigid support element 370. An opening or incision 250 is made in the skin 260 of patient 270 to provide access for introducer sheath 300 and dilator 200 through artery wall 280 into artery 290.

FIG. 7(b) illustrates the improved introducer sheath of FIG. 4(b) during insertion and at the time of engagement with incision 250 and skin 260. Dilator tip 210 is inserted into the incision 250 to thereby expand the opening enough for introducer sheath 100 to access artery interior 290. During insertion, outer layer 240 contacts skin 260 and the internal surfaces of incision 250. Semi-rigid support element 370 stabilises the position of outer layer 240 longitudinally wherein the friction against outer layer 240 caused during the passage of dilator 200 through opening or incision 250 is resisted by semi-rigid support element 370 thereby minimising the crumpling, compressing, or folding of the outer layer 240 in the direction from the distal opening 220 towards the proximal end (not shown). This prevents the exposure of the inner layer 230 and the sharp edges that typically form at the distal opening which catch and cause damage to the incision 250 or artery wall 280 during insertion.

FIG. 7(c) illustrates the improved introducer sheath of FIG. 4(b) during deeper insertion into artery 290. Inner layer 230 is minimally exposed whilst in artery 290 and its sharp edges are protected by outer layer 220. The likelihood of failed insertion is reduced, however, even in the events of removal and reinsertion, the likelihood of damage to the vessel is reduced.

The problem illustrated in FIGS. 7(a) to 7(c) also applies to single layer introducer sheaths exemplified in FIG. 4(a). While there is no potential for exposure of an inner layer, the crumpling or folding of the single layer may lead to a sub-optimal insertion, blockage, and subsequent failure condition, and therefore the semi-rigid support element 370 similarly reduces risk.

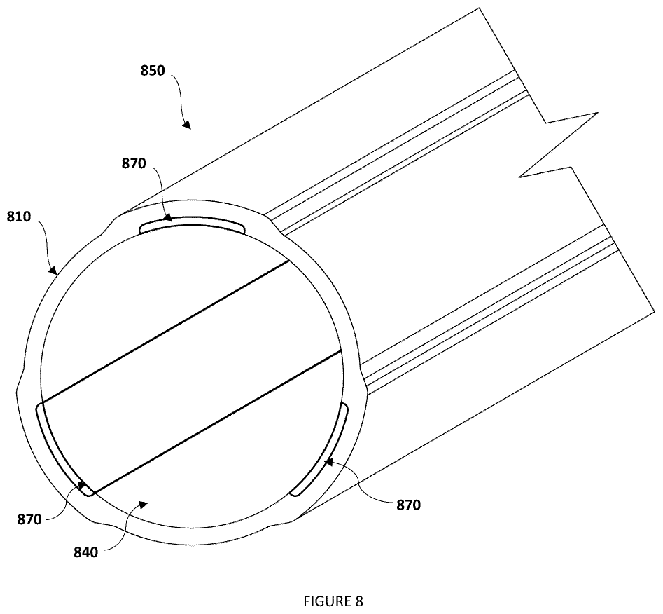

FIG. 8 illustrates an alternative construction of the improved introducer sheath whereby outer elastomeric tube 850 with a lumen 840 therethrough is constructed of elastomeric layer 810 and three semi-rigid support elements 870. The three semi-rigid support elements 870 are equally spaced about the substantially circular profile of outer elastomeric tube 810 and are constructed as per semi-rigid support element 870. Outer elastomeric tube 850 is constructed in all other respects per outer elastomeric tube 850. However, the construction of FIG. 8 may be preferred in instances where the materials selected for the construction of the elastomeric layer 810 are very thin or very soft. Such materials are generally preferred for insertion into small vessel, for instance, in ophthalmic microsurgeries.

Elastomeric layer 810 is shaped to receive each semi-rigid support element 870 such that the resulting outer elastomeric tube 850 is formed from the assembled elastomeric layer 810 and semi-rigid support elements 870, which maintain the expanding inner layer within.

Each semi-rigid support element 870 is positioned in-line with the internal circular profile of outer elastomeric tube 850 such that the inner surface of outer elastomeric tube 850 is unabridged and complete. The outer profile of the exterior surface of the outer elastomeric tube 850 is shaped by the profile of each semi-rigid support element 870 such that the outermost surface of each semi-rigid support element 870 is covered by elastomeric layer 810 at a similar thickness to the remainder of outer elastomeric tube 870.

For particularly sensitive insertions, the outermost surface of outer elastomeric tube 850 is of uniform thickness, such that portions of elastomeric layer 810 are thicker between each of the semi-rigid support elements 870 and are thinner where they extend over each semi-rigid support element 870, to provide an outermost and innermost surface of outer elastomeric tube 870 that is substantially smooth, circular and uniform.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be understood that the terms ‘fastener’ or ‘fastening’, ‘coupling’ or ‘sealing’ when used alone or together with other terms such as ‘means’ or others, may be used interchangeably where interpretation of the term would be deemed by persons skilled in the art to be functionally interchangeable with another. Further, the use of one of the aforementioned terms does not preclude an interpretation when another term is included.

The various apparatuses and components of the apparatuses, as described herein, may be provided in various sizes and/or dimensions, as desired. Suitable sizes and/or dimensions will vary depending on the specifications of connecting components or the field of use, which may be selected by persons skilled in the art.

It will be appreciated that features, elements and/or characteristics described with respect to one embodiment of the disclosure may be used with other embodiments of the invention, as desired.

Although the preferred embodiments of the present disclosure have been disclosed for illustrative purposes, those skilled in the art will appreciate that various modifications, additions, and substitutions are possible, without departing from the scope and spirit of the disclosure and accompanying claims.

It will be understood that when an element or layer is referred to as being “on” or “within” another element or layer, the element or layer can be directly on or within another element or layer or intervening elements or layers. In contrast, when an element is referred to as being “directly on” or “directly within” another element or layer, there are no intervening elements or layers present.

As used herein, the term “and/or” includes any and all combinations of one or more of the associated listed items.

It will be understood that, although the terms first, second, third, etcetera, may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present disclosure.

Spatially relative terms, such as “lower”, “upper”, “top”, “bottom”, “left”, “right” and the like, may be used herein for ease of description to describe the relationship of one element or feature to another element(s) or feature(s) as illustrated in the figures. It will be understood that spatially relative terms are intended to encompass different orientations of structures in use or operation, in addition to the orientation depicted in the drawing figures. For example, if a device in the drawing figures is turned over, elements described as “lower” relative to other elements or features would then be oriented “upper” relative the other elements or features. Thus, the exemplary term “lower” can encompass both an orientation of above and below. The device may be otherwise oriented (rotated 90 degrees or at other orientations) and the spatially relative descriptors used herein should be interpreted accordingly.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the description are described herein with reference to diagrams and/or cross-section illustrations, for example, that are schematic illustrations of preferred embodiments (and intermediate structures) of the description. As such, variations from the shapes of the illustrations as a result, for example, of manufacturing techniques and/or tolerances, are to be expected. Thus, embodiments of the description should not be construed as limited to the particular shapes of components illustrated herein but are to include deviations in shapes that result, for example, from manufacturing.

Unless otherwise defined, all terms (including technical and scientific terms) used herein have the same meaning as commonly understood by one of ordinary skill in the art to which this description belongs. It will be further understood that terms, such as those defined in commonly used dictionaries, should be interpreted as having a meaning that is consistent with their meaning in the context of the relevant art and will not be interpreted in an idealised or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the description. The appearances of such phrases in various places in the specification are not necessarily all referring to the same embodiment. Further, when a particular feature, structure, or characteristic is described in connection with any embodiment, it is within the purview of one skilled in the art to effect and/or use such feature, structure, or characteristic in connection with other ones of the embodiments.

Embodiments are also intended to include or otherwise cover methods of using and methods of manufacturing any or all of the elements disclosed above.

While the invention has been described above in terms of specific embodiments, it is to be understood that the invention is not limited to these disclosed embodiments. Upon reading the teachings of this disclosure many modifications and other embodiments of the invention will come to the mind of those skilled in the art to which this invention pertains, and which are intended to be and are covered by both this disclosure and the appended claims.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It is indeed intended that the scope of the invention should be determined by proper interpretation and construction of the appended claims and their legal equivalents, as understood by those skilled in the art relying upon the disclosure in this specification and the attached drawings.

1-19. (canceled)

20. A system for protecting a luminal surface of a blood vessel, the system including an introducer sheath comprising:
an annular collar defining an opening extending through the collar,
the annular collar having attached thereto a circumferentially retractable elongated sleeve attached at a proximal opening of the elongated sleeve and extending longitudinally to define a luminal channel terminating at a distal opening of the elongated sleeve,
the elongated sleeve comprising a circumferentially continuous elastomeric outer tube extending longitudinally and spanning the entire length of the sleeve;
wherein the circumferentially continuous elastomeric outer tube has a smooth inner surface with a substantially round cross-section and comprises one or more support elements extending longitudinally from the distal opening towards the proximal opening.

21. The system according to claim 20, wherein the inner surface of the elastomeric outer tube is at least partially formed by the one or more support elements, preferably wherein the one or more support elements form less than 50% of the inner surface, more preferably less than 25%.

22. The system according to claim 20, wherein the outer surface of the elastomeric outer tube is formed by an elastomeric layer.

23. The system according to claim 20, wherein the elongated sleeve comprises a coiled expandable inner sheet positioned longitudinally within a lumen formed by the circumferentially continuous elastomeric outer tube, positioned substantially in contact with a luminal surface of the circumferentially continuous elastomeric outer tube.

24. The system according to claim 20, wherein the one or more support elements extend longitudinally, span the entire length of the sleeve and are bonded to an inner luminal surface of the circumferentially continuous elastomeric outer tube, wherein the one or more support elements are configured to resist longitudinal compression of the circumferentially continuous elastomeric outer tube along the entire length of the sleeve.

25. The system according to claim 20, wherein the one or more support elements are formed of a biocompatible material and have a k value approximately equal to or greater than 0.1, wherein k is expressed by the formula:
k=F/δ/l, wherein:
k=longitudinal stiffness in Newtons per millimeter per meter (N/mm/m)
F=applied longitudinal force in Newtons (N)
δ=extension, deflection in millimeters (mm)
l=length of support element in meters (m).

26. The system according to claim 20, wherein the circumferentially continuous elastomeric outer tube is formed of a biocompatible material and has a kr value approximately equal to or greater than 1.5, wherein kr is expressed by the formula:
kr=ki/k0=(F*δ0)/(F*δi), wherein:
kr=stiffness ratio of improved elastomeric layer to original
k0=original elastomeric layer longitudinal stiffness (N/mm/m)
ki =improved elastomeric layer longitudinal stiffness (N/mm)
F=applied longitudinal force (N)
δ0=original elastomeric layer extension, deflection (mm)
δi=improved elastomeric layer extension, deflection (mm).

27. The system according to claim 20, wherein the one or more support elements comprises a material selected from the group consisting of fibrous materials, textiles, metals, metal alloys, polymers, mixtures or composites thereof, the selected material having at least 50 times the modulus of the continuous elastomeric outer tube.

28. The system according to claim 20, wherein the one or more support elements are formed from a combination of a wire or strip consisting of any one of stainless steel, nitinol, or mixture or composite thereof, and a crescent shaped polymeric material.

29. The system according to claim 20, further comprising a dilator having a dilator tip of greater diameter than the distal opening of the elongated sleeve, wherein the dilator tip is configured to pass through the lumen of the circumferentially retractable elongated sleeve.

30. A method comprising the steps of:
obtaining an introducer sheath comprising:
an annular collar defining an opening extending through the collar, the annular collar having attached thereto a circumferentially retractable elongated sleeve attached at a proximal opening of the elongated sleeve and extending longitudinally to define a luminal channel terminating at a distal opening of the elongated sleeve,
the elongated sleeve comprising a circumferentially continuous elastomeric outer tube extending longitudinally and spanning the entire length of the sleeve, wherein the circumferentially continuous elastomeric outer tube has a smooth inner surface with a substantially round cross-section and comprises one or more support elements extending longitudinally from the distal opening towards the proximal opening;
obtaining an expandable inner sheet; and
positioning the expandable inner sheet longitudinally within a lumen formed by the circumferentially continuous elastomeric outer tube and positioned substantially in contact with a luminal surface of the circumferentially continuous elastomeric outer tube;
whereby the one or more support elements are configured to resist longitudinal compression of the circumferentially continuous elastomeric outer tube.

31. The method according to claim 30, comprising the steps of:
obtaining a dilator having a dilator tip of greater diameter than the distal opening of the elongated sleeve, and
passing the dilator tip through a lumen formed by the expandable inner sheet and along the length of the circumferentially retractable elongated sleeve.

32. The method according to claim 30, comprising the step of:
passing the dilator tip and introducer sheath through a blood vessel wall.

33. A system for protecting a luminal surface of a blood vessel, the system including:
an introducer sheath comprising:
an elastomeric outer tube having a smooth and continuous inner surface along its length and including one or more longitudinally extending stiffening ridges on its outer surface, wherein each of the stiffening ridges comprises a support element, and
a coiled expandable inner sheet positioned within a lumen of the elastomeric outer tube; and
a dilator having a dilator tip of greater diameter than a distal opening of the elastomeric outer tube, wherein the dilator tip is configured to pass through a lumen of the coiled expandable inner sheet.

34. The system according to claim 33, wherein the introducer sheath further comprises an annular collar formed of a rigid material and having attached thereto a proximal opening of the elastomeric outer tube, the annular collar being configured to allow a user to introduce materials into the lumen of the elastomeric outer tube.

35. The system according to claim 33, wherein the inner surface of the elastomeric outer tube is at least partially formed by the one or more support elements.

36. The system according to claim 33, wherein each of the one or more stiffening ridges comprises a longitudinally extending radial protrusion of the elastomeric outer tube.

37. The system according to claim 33, wherein the introducer sheath is a flexible structure tapering, at least in part, between the proximal end and the distal end.

38. The system according to claim 33, wherein a section of the support element is thicker in the middle than at its ends.

39. The system according to claim 33, wherein the one or more support elements comprises a material having at least 50 times the modulus of the elastomeric outer tube.
